(19) United States
(12) Patent Application Publication
(10) Pub. No.: US 2013/0211208 A1
(43) Pub. Date: Aug. 15, 2013

(54) SMART MATERIALS, DRY TEXTILE SENSORS, AND ELECTRONICS INTEGRATION IN CLOTHING, BED SHEETS, AND PILLOW CASES FOR NEUROLOGICAL, CARDIAC AND/OR PULMONARY MONITORING

(71) Applicants: Vijay K. VARADAN, Fayetteville, AR (US); Pratyush Rai, Fayetteville, AR (US); Prashanth Shyam Kumar, Fayetteville, AR (US); Gyanesh N. Mathur, Fayetteville, AR (US); M. P. Agarwal, State College, PA (US)

(72) Inventors: Vijay K. VARADAN, Fayetteville, AR (US); Pratyush Rai, Fayetteville, AR (US); Prashanth Shyam Kumar, Fayetteville, AR (US); Gyanesh N. Mathur, Fayetteville, AR (US); M. P. Agarwal, State College, PA (US)

(21) Appl. No.: 13/657,854

(22) Filed: Oct. 22, 2012

Related U.S. Application Data
(63) Continuation of application No. 13/415,698, filed on Mar. 8, 2012, now abandoned.
(60) Provisional application No. 61/450,423, filed on Mar. 8, 2011.

(51) Int. Cl. A61B 5/00; A61B 5/0478; A61B 5/01; A61B 5/0496; A61B 5/0205; A61B 5/0408; A61B 5/0492

(52) U.S. Cl. CPC A61B 5/6804; A61B 5/0075; A61B 5/0408; A61B 5/0478; A61B 5/0492; A61B 5/0496; A61B 5/02055; A61B 5/015; A61B 5/0002
USPC 600/301; 600/300

(57) ABSTRACT

Sensors mounted on a textile include at least one of electrically conductive textile electrodes; single or multiple optically coupled infrared and red emitter and photodiode or photo transistor; and thin film or Resistive Temperature Detector (RTD). Textile electrodes, electrical connections, and electrical functionalization use at least one of nanoparticles, nanostructures, and mesostructures. Conductive thread, for electrical connections, may include a fiber core made from conductive materials such as but not limited to metals, alloys, and graphine structures, and a sheath of insulating materials such as but not limited to nylon, polyester, and cotton.

### CROSS-REFERENCE TO RELATED APPLICATION

This application claims the benefit of priority under 35 U.S.C.§119(e) of U.S. Provisional Patent Application No. 61/450,423, filed Mar. 8, 2011, the disclosure of which is incorporated herein by reference.

### TECHNICAL FIELD

The present disclosure relates to electronic and optical sensor technologies, and their packing to enable their integration into textile. These sensor capabilities will enable the use of textile for health monitoring, while operating in contact or in proximity of person's body.

### BACKGROUND

Chronic disease management and in-hospital patient care are two major contributors to healthcare costs. The former consists of patients in need of repeated tests to assess disease progression or protocols for drug dosage adjustments. The latter consists of patients recovering from surgeries or in need for constant observation for diagnosis. They contribute to approximately 30% ($690 billion) and 20% ($460 billion) of the annual healthcare costs, respectively, in the United States of America. See, e.g., Tabibiazar R., Edelman S. V., “Silent Ischemia in People With Diabetes: A Condition That Must Be Heard,” Clinical Diabetes, Vol. 21 (1), 5-9 (2003), the disclosure of which is incorporated herein by reference.

Cardiovascular diseases and neurological disorders form the majority of diseases that need constant or periodic medical attention. The concept of continuous health monitoring can be translated as point of care technology for preventive/corrective medicine and as metabolic rate estimation and regulation as a part of healthy lifestyle. Point of care technology aims at enabling diagnostics in hospice, at home or ambulatory (on the move).

Health monitoring textile is a type of wearable and ambient healthcare technology: an ensemble of non-invasive sensor systems, which operates in contact or in proximity of person's body. Resemblance to a conventional wearable item (apparel) or integrability in it increases the relevance of such a device. Wearable fabric based items like vests, socks, shorts, head bands, arm bands, wrist bands and caps, foot wear, and drapes like bed spreads/sheet and pillow covers can incorporate sensors for monitoring the health of an individual for diabetes, neurological, and cardiovascular monitoring.

Neurological disorders such as sleep disorders and sleep deprivation affect more than thirty million people, while another six million have moderate to severe sleep apnea in which breathing briefly stops. That is nearly one in five Americans, making sleep apnea as prevalent as asthma or diabetes. More than six million people have restless leg syndrome and periodic limb movement disorder which jolts them awake repeatedly. As many as twenty-five million people remain undiagnosed and untreated which will account for over $22 billion in unnecessary health care costs. Apart from physical factors such as obesity, studies have shown that the cumulative long-term effects of sleep loss and sleep disorders are associated with a wide range of serious health consequences and many life threatening illnesses including increased risk of hypertension, diabetes, depression, heart attack, impotence and stroke, to name a few. In addition, a significant percentage of severe traffic and industrial accidents may be caused by the involuntary human transition from wakefulness to sleep.

There are also apparent links between deficits in brain chemistry and obstructive sleep apnea (OSA) and REM sleep behavior disorder (RBD). Both are relatively common sleep problems that disturb the slumber and daytime behavior of millions of Americans. It has been reported that multiple system atrophy (MSA), a rare and fatal degenerative neurological disease, is almost always accompanied by severe sleep disorder. Patients with the fewest dopamine-producing neurons in the striatum of their brains had the worst RBD symptoms, talking and violent flailing during their sleep. People with OSA show tissue loss in brain regions that help store memory, thus linking OSA with memory loss and Alzheimer's disease. Obstructive sleep apnea, in which breathing temporarily stops during a person's sleep, often affects adults but goes undiagnosed in many cases. Its most notable symptoms are snoring and excessive daytime sleepiness, though it can also affect blood pressure, memory and even reaction-time while driving.

What is needed is a robust and non-disruptive monitoring bed sheets- and pillow cases-based system that addresses continuous biopotential measurements, which can analyze and record the required parameters while the patient is at home and sleeping in his or her own bed.

Textiles offer a durable platform for embedded sensor and communication systems, with the components like sensors and communication chip-sets stitched or woven into the fabric. Individual electronic components can be mounted on the textile and connected through electrical connects that have been built in or manufactured in the textile itself. The electronic functionality should be embedded while maintaining the textile properties of product like wearing comfort and durability. Manufacturing techniques used for such smart textiles have to be compatible with existing textile manufacturing techniques to minimize additional costs.

Physiological signals, such as but not limited to, Electrocardiogram (ECG), Pulse rate (and heart rate variability), blood pressure, Electroencephalography (EEG), electro-oculography (EOG) and electromyography (EMG), provide a comprehensive medical status of a person. In combination with wireless communication technology, they can be used for remote medical diagnosis or prognosis. Textile based dry electrodes with lower electrode-skin contact impedance for improved performance in bioelectric signal acquisition is important to achieve un-obstructive and long term health monitoring. This is not possible with conventional wet electrodes due to drying of the conductive gel over period of time that leads to loss of functionality and skin irritation. Un-obstructive blood pressure monitoring requires an alternative to the conventional inflatable cuff based sphygmomanometer. Also, such a setup is difficult to incorporate in textile and very energy intensive for mobile health monitoring.

Printing processes can be used for making complex high resolution designs on a wide range of substrate, including textile. See, e.g., Sherman, R., “Could Printed Electronics Replace Traditional Electronics?” Printed Circuit Design & Fab, 27 (3), 38, 40, 42 (2010), the disclosure of which is incorporated herein by reference. Printing allows for direct pattern transfer of electronics with little or no waste of material and thus a cost effective alternative to photolithography techniques. Among the popular printing technologies, screen printing and gravure are well suited for mass produced electronics on textile because of their parallel printing technology and the substrate handling. See, e.g., Sheats, J., R., Biesty, D., Noel, J., Taylor, G., N., “Printing technology for ubiquitous electronics,” Circuit World, 36 (2), 40-47 (2010); Kah, B., E., “Printing methods for printed electronics,” 24th International Conference on Digital Printing Technologies. Digital Fabrication 2008, 15-20 (2008), the disclosures of which are incorporated herein by reference.

Parallel printing, as compared to serial printing technologies like ink jet printing, has a higher manufacturing throughput. Screen printing and gravure printing technologies do not deviate significantly from garment making techniques making them cost effective. These technologies will enable fabrication (over a large surface area) of electronics with varied functionality like:—sensor systems and flexible printed circuits for electrical connections between sensors and the embedded wireless telemetry systems.

The textile based healthcare applications and packaging technology described in this section aim at improved sensor performance and seamless integration of the sensor systems in the textile for un-obstructive health monitoring. The technologies use a novel combination of nanomaterials and textile fabric for sensor and packaging electronics.

### SUMMARY

According to various aspects of the disclosure, sensors mounted on a textile include at least one of electrically conductive textile electrodes; single or multiple optically coupled infrared and red emitter and photodiode or photo transistor; and thin film or Resistive Temperature Detector (RTD).

According to the disclosure, textile electrodes, electrical connections, and electrical functionalization use at least one of nanoparticles, nanostructures, and mesostructures.

In accordance with some aspects of the disclosure, conductive thread, for electrical connections, may include a fiber core made from conductive materials such as but not limited to metals, alloys, and graphine structures, and a sheath of insulating materials such as but not limited to nylon, polyester, and cotton.

Further advantages and embodiments are apparent from the appended drawings.

### BRIEF DESCRIPTION OF THE DRAWINGS

In the figures:

FIG. 1 illustrates a design of an exemplary arm band for brachial artery plethysomography in accordance with various aspects of the disclosure;

FIG. 2 illustrates an exemplary mechanism for adjusting the relative positions of the sensors according to various aspects of the disclosure;

FIG. 3 illustrates an exemplary packaging technology for the sensors in accordance with various aspects of the disclosure;

FIG. 4 is a graph showing the signal from an exemplary 3-Lead ECG dry textile electrodes system with leads 1, 2, and 3 plotted simultaneously;

FIG. 5 is a graph of a leading electrocardiograph (lead 2) and lagging brachial artery pulse data acquired on the same time line for measurement of pulse transit time;

FIG. 6 is a graph showing calibration curves for systolic and diastolic blood pressures versus pulse transit time;

FIG. 7 is a graph illustrating a Resistive Temperature Detector based temperature sensor calibration curve; and

FIG. 8 is a graph of brain rhythm such as Alpha, Beta,

Theta and Delta waves from a textile sensor in pillow cases and bed sheets.

### DETAILED DESCRIPTION

The electrode design for electrophysiological sensing (ECG, EEG, etc.) is developed as a electrically functionalized piece of fabric mounted on a spring mechanism. The electrode fabric is dyed with conductive ink, or enmeshed/decorated with conductive nanoparticles. The electrode surfaces have been engineered to have nanoscale and mesoscale free standing conductive structures. This is done to increase the effective surface area of the electrodes. Electrode surface area, which is in contact with the skin, is important to the signal quality. The signal measured is electric potential across the load resistance between the two electrodes that can be conceived as the impedance due to body bulk, skin and electrodes. Large electrode surface area results in low skin-electrode contact resistance. The free standing structures are deposited on the above mentioned conductive fabric by flocking electrically conductive fibers. Another technique is printing the electrodes with nanocomposite ink, which will have nanostructures on the surface of the printed thick film for increase surface area. Printed electrode for Electrocardiography (ECG, EEG etc.) is a technology based on the fabric itself. The electrodes system printed on the textile serves for multi-lead ECG signal acquisition, when the electrode surface is in contact with person's skin. The composition of the ink will be described in more detail below.

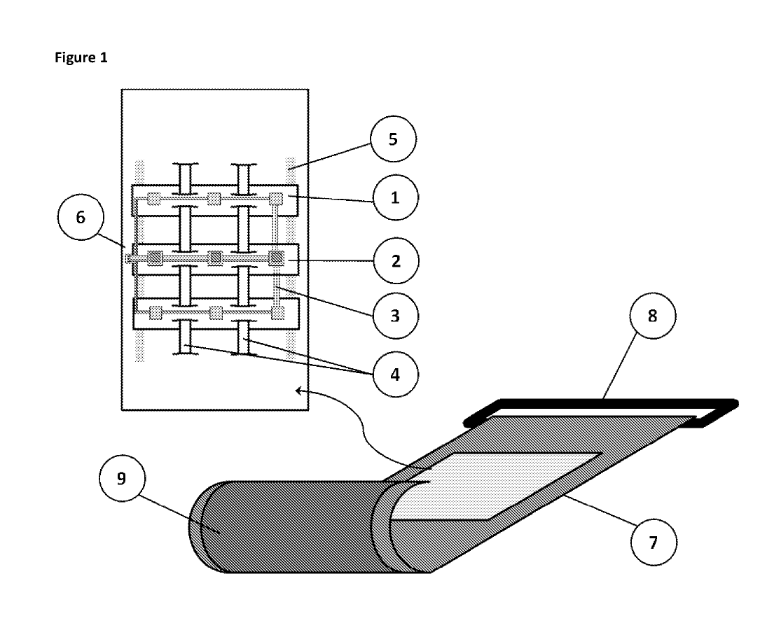

FIG. 1 illustrates the design of an arm band for brachial artery plethysomography. The optically coupled infrared emitters and photodiode have been arranged in arrays and connected to a breakout plug that can be connected to the primary circuit on the textile. Blood pressure measurement system is an opto-electronic system, plethysmograph (PPG) that monitors the blood flow in the brachial artery in the left arm. A multichannel infra red emitter-detector (FIG. 1) system is placed on the left on the axis of the brachial artery (inside part of the left arm) to detect change in blood flow of the brachial artery. The system is used in combination with the ECG measurement to estimate the time it takes for the pulse, pulse transit time (PTT) to move from the aortic valve to the PPG site. The PTT is an index for estimation of arterial blood pressure (ABP). The PPG system uses infrared reflectance by the blood for monitoring the blood flow volume. Positions for the emitter arrays 1 and the detector array 2 are important to get the optimum reflectance signature.

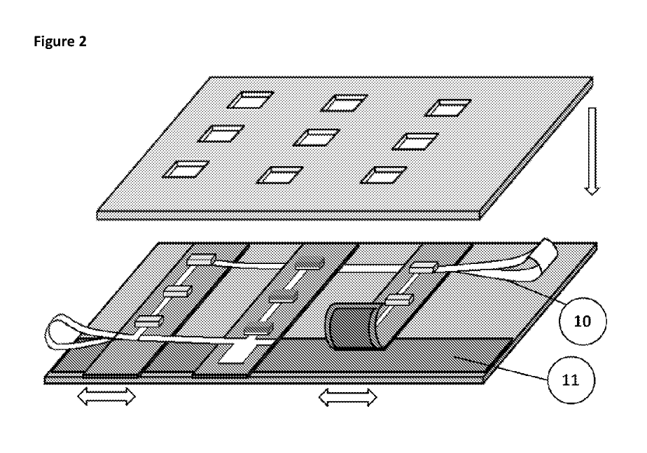

FIG. 2 illustrates the mechanism for adjusting the relative positions of the sensors. The sensors positions can be changed by sliding the emitter arrays on a spin and securing them by hock and loop to accommodate for different arm diameters. The size of the arm varies from person to person. To address this issue, provision for adjusting the array spacing has been provided. The emitters and detectors are surface mounted devices (SMDs). (FIG. 2) They have been soldered on to a flexible printed circuit 3, 10 with flat flexible connections running between components. This is to enable packaging of the components in a textile based arm band. The components are arranged in three arrays. Array in the middle is stationary, while the flanking arrays can move on two spines 4. The system uses hook and loop arrangement 5, 11 to secure the arrays in position. The band system has been designed as a detachable component of the textile health monitoring system. A flat flexible connection port 6 is provided on the band for connection to ancilliary or master circuit for power supply and signal relay. The use of flexible printed circuit is to enable packaging of the components in a textile based arm band 7 with a buckle 8, and hock and loop 9 for strapping around the arm.

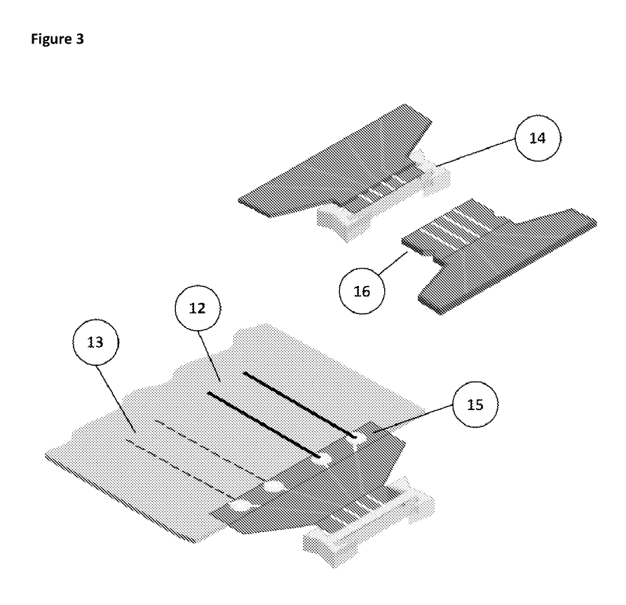

FIG. 3 illustrates the packaging technology for the sensors. The textile based electric connection lines for the sensors are linked to the break out pins of a socket. The figure depicts the socket with thread as well as printed lines on fabric. The corresponding plug is mounted on the electronics for wireless communication and power supply. Similar concept is used for connecting the arm band electronics to the master circuit. Printed electrical connects, on the textile fabric, can function like a flexible textile based printed circuit film. This will act as a system to facilitate packaging of the sensor systems, and amplifier-transmitter electronics in the textile. (FIG. 3) The connect lines or conductive traces 12 use nanomaterial composite based inks. The binder itself can serve as printing ink, so that the conductive traces can be insulated by an overlay of traces made with binder only. The ink formulation uses modified acrylic, epoxy or resin binders with conductive nano particles and nanostructures dispersed in it. The nanocomposite based conductive patterns provide electrical properties similar to conductive metal wires or strips, while being able conform with the flexibility of textile. Binder's adhesion properties allow for printing on nylon, cotton, lycra, spandex, neoprene or other elastomeric fabric or film. The binder possesses high elasticity; therefore, it will protect the traces from disruption due to stretching of the fabric or film.

Textile based connections for packaging of sensor and wireless electronics in textiles, can be accomplished with conductive threads 13. The textile health monitoring system also uses conductive threads made of conductive fiber core and an insulation sheath. Conductive fiber core can be made of metals like silver, copper, titanium; alloys like stainless steel, nickel-cromium; and graphine structures like carbon nanotubes. The sheath can be made of nylon, polyester, and cotton. These threads are compatible with machine weaving. In addition to being compatible with textile platform, the printed connections and conductive threads are resistant to triboelectric effect. This prevents build up of static charge, which occurs when wearing textile products. Thus, signal artifacts due to static charge build up are avoided.

The printed connections and conductive thread connections are required to be able to connect to the electronics for wireless communication and power supply. While these components are not made on textile substrate, their electronic connects do not readily interface with the textile based connects. The textile health monitoring system uses a special electronic connector assembly (FIG. 3), which houses a socket 14 with break out pins attached to corresponding textile connects 15 with rivets, crimps or silver epoxy. The socket is compatible to the plug 16 on the electronic module for wireless communication and power supply.

### Wireless Communication Platform:

Coupled with a low power microcontroller and Bluetooth module (Zigbee, WiFi and other communication protocols as appropriate), the sensor data can be streamed to commercial off-the-shelf cell phones and smart phones, laptops, computer, and handhelds units. A software system has also been developed for cellular ‘smartphones’ that can collect sensor data over Bluetooth and can relay data over 3G, Wi-Fi, WiMax or any outgoing connection with RFID. Apart from the cost benefits of using an off-the-shelf cell phone for data relaying, our software system will provide two other distinguishing features. First, it will implement filtering algorithms on the cell phone to mitigate issues due to motion and other artifacts, rendering clean data. It will provide a visualization interface at the cell phone through which users can see salient features of their heart activity such as heart rate. The software on the phone will run simple machine learning algorithms to perform preliminary anomaly detection. In case of an emergency, it can either alert the user and recommend him/her to hospital locations near his/her present location or make an automated call to the patient's physician with his/her present location. Thus caregivers can access into vital information anywhere and at anytime within the healthcare networks. The Zigbee based WiFi system used is capable of handling 65,000 patients at a given time.

The geo-tagged data is transferred to a cloud cluster and stored in a secure database and SD card. For physician diagnostics we will provide a new backend service, where the doctor can log into our system and can visually look at past ECG, EEG and other related data from the user or real-time continuous data (whichever is deemed necessary). If the physician desires, he/she can use our machine learning services to detect anomalies in the data that was collected in the past. In the event that our machine learning algorithms detect abnormalities in the data, our VoIP service can make phone calls or send SMS messages to physicians.

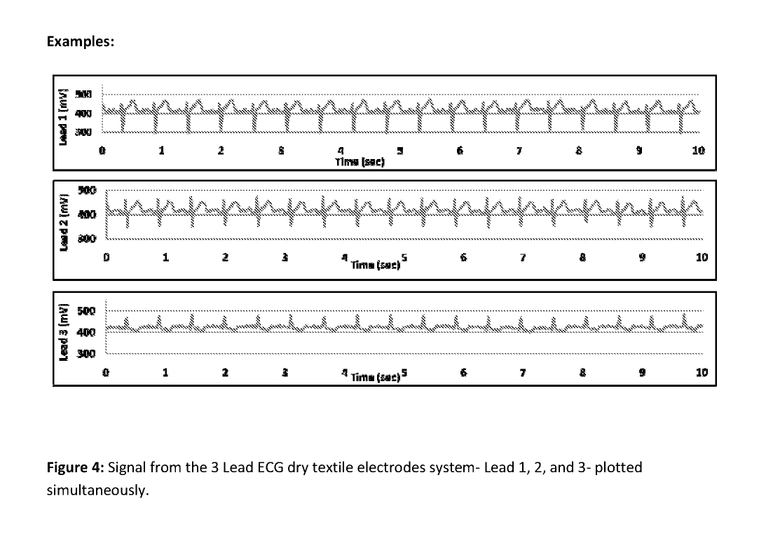

The example presented in FIG. 4 is to illustrate the ability of the smart textile health monitoring system to acquire 3 lead ECG signal using dry textile electrodes. ECG acquired here are Lead 1- between augmented right arm and augmented left arm, Lead 2- between augmented right arm and augmented left leg, and Lead 3- between augmented left leg and augmented left arm. This basic form of ECG acquisition monitors the atrial activity and ventricular activity of the heart. The data is also used for heart rate calculation and arterial blood pressure estimation.

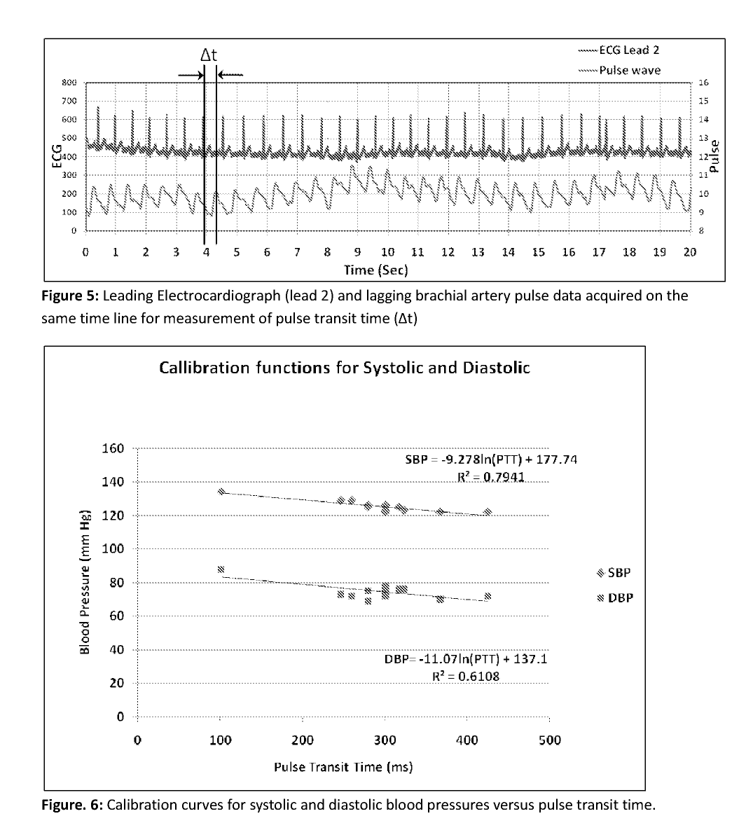

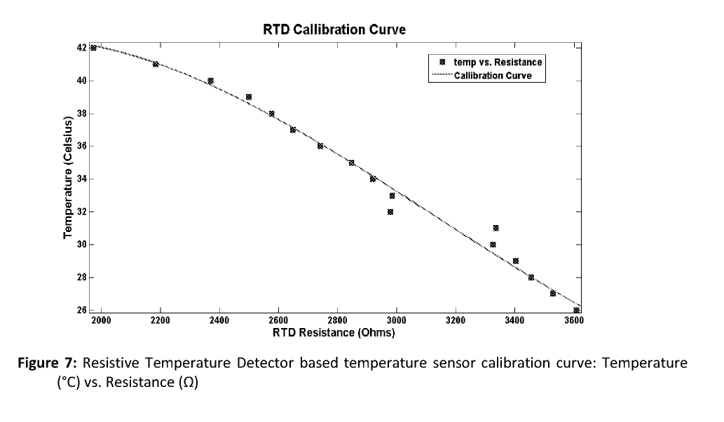

The example presented in FIGS. 5 and 6 illustrates the blood pressure estimation application. While FIG. 5 shows the concept behind calculation of the pulse transit time (PTT), FIG. 6 shows the calibration curves used as the transducer functions for estimation of atrial systolic and diastolic blood pressure from PTT.

The example presented in FIG. 7 illustrates the body temperature sensing application of the flexible thin film temperature sensor. The calibration curve is used as a function by the signal acquisition software for converting the change in resistance of the thin film channel to temperature. The range of linear response is 32° C. to 38° C., which is the range of the temperatures observed at the axilliary location of the arm. The axial temperature range that covers from normal condition to feverish. See, e.g., Lodha, R., Mukerji, N., Sinha, N., Pandey, R., M., and Jain, Y., “Is Axillary Temperature an Appropriate Surrogate for Core Temperature?” Indian Journal of Pediatrics, 67 (8), 571-574 (2000), the disclosure of which is incorporated herein by reference.

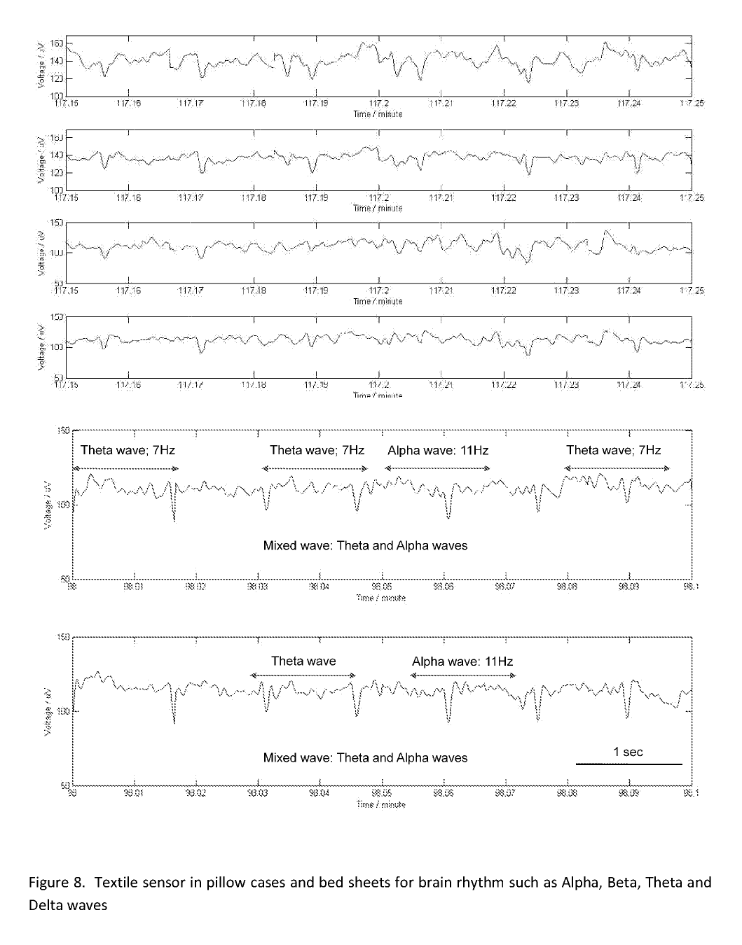

The examples presented in FIG. 8 are typical brain rhythm as measured by the textile based sensor system. They are consistent with the regular wet gel electrodes used in the hospital. See, e.g., Allan Rechtschaffen and A. Kales, A manual of standardized terminology, techniques and scoring system for sleep stages of human subjects, Brain Information Service/Brain Research Institute, University of California, Los Angeles, Calif. (1977), the disclosure of which is incorporated herein by reference.

It will be apparent to those skilled in the art that various modifications and variations can be made to the smart materials, dry textile sensors, and electronics integration in clothing, bed sheets, and pillow cases of the present disclosure without departing from the scope of the invention. Throughout the disclosure, use of the terms “a,” “an,” and “the” may include one or more of the elements to which they refer. Other embodiments of the invention will be apparent to those skilled in the art from consideration of the specification and practice of the invention disclosed herein. It is intended that the specification and examples be considered as exemplary only.

What is claimed is:

1. Sensors mounted on a textile, comprising at least one of:
a. electrically conductive textile electrodes;
b. single or multiple optically coupled infrared and red emitter and photodiode or photo transistor; and
c. thin film or Resistive Temperature Detector (RTD).

2. Textile electrodes, electrical connections, and electrical functionalization using at least one of nanoparticles, nanostructures, and mesostructures.

3. A device according to claim 2, wherein the nanoparticles, nanostructures and mesostructures comprise ink suspensions in organic binders.

4. A device according to claim 3, wherein the ink suspensions in organic binders include acrylic, epoxy, heat curable, and photo curable resins.

5. A device according to claim 3, wherein the ink suspensions are compatible with textile compatible pattern transfer techniques.

6. A device according to claim 5, wherein the patter transfer techniques include screen printing, stamping, ink jet, and gravure.

7. A device according to claim 2, wherein the nanoparticles, nanostructures and mesostructures are deposited on textile.

8. A device according to claim 2, wherein the nanoparticles, nanostructures and mesostructures are deposited on textile by at least one of flocking and dyeing.

9. A device according to claim 1, wherein the sensors include said textile electrodes for sensor-actuator applications.

10. A device according to claim 9, wherein the sensor-actuator applications include Electrocardiography (ECG), Impedance Cardiography (ICG), Electroencephalography (EEG), Electromyography (EMG), and Electrooculography (EOG).

11. A device according to claim 1, wherein the sensors are mounted on an arm band, for monitoring pulsatile blood flow in major arteries of the body.

12. A device according to claim 11, wherein the position of the sensors can be adjusted based on the diameter of wearer's arm.

13. A device according to claim 1, wherein the sensors are configured to monitor thermal distribution in a person's body.

14. A device according to claim 2, wherein said electrical connections comprise conductive thread, said conductive thread comprising:
a fiber core made from at least one conductive material; and
a sheath made from at least one insulating material.

15. The device of claim 14, wherein the conductive material comprises at least one of metals, alloys, and graphine structures, and the insulating material comprises at least one of nylon, polyester, and cotton

16. A device according to claim 5, wherein electrical connections, resistant to triboelectric effect, for sensor signal relay are printed with said ink suspensions.

17. A device according to claim 10, wherein electrical connections, resistant to triboelectric effect, for sensor signal relay are made of said conductive threads.

18. A device according to claim 2, further comprising an electrical connector assembly for connecting said electrical connections to an electronics module.

19. A device according to claim 10, further comprising an electrical connector assembly for connecting said conductive thread connections to an electronics module.

20. A textile comprising:
at least one sensor enmeshed in the textile, the sensor comprising a textile-based or textile-integrable sensor;
at least one textile-based or textile-integrable amplifier-transmission module configured to be electrically coupled with the sensors; and
a textile-based or textile-integrable power supply configured to be electrically couple with at least one of the sensor and the amplifier-transmission module.

21. The textile of claim 20, wherein the textile comprises a garment configured to be worn by a user.

22. The textile of claim 21, wherein the textile houses one of:
a. connective lines of conductive fiber with a metallic or alloy core for supporting at least one of electrical, optical, opto-electronic, and electro-mechanical medical monitoring devices on or in the vicinity of the user wearing the garment;
b. connective lines of conductive fiber with metallic or non-metallic conductive nanoparticles blended or decorated fabric for supporting at least one of electrical, optical, opto-electronic, and electro-mechanical medical monitoring devices on or in the vicinity of the user wearing the garment;
c. connective lines of conductive filament with a metal coated fiber or alloy-fiber blend core for supporting at least one of electrical, optical, opto-electronic, and electro-mechanical medical monitoring devices on or in the vicinity of the user wearing the garment; and
d. connective lines of optic fiber for supporting at least one of electrical, optical, opto-electronic, and electro-mechanical medical monitoring devices on or in the vicinity of the user wearing the garment.

23. The textile of claim 22, wherein the garment is washable.

24. The textile of claim 20, further comprising a system for communicating signals from said at least one sensor to a computational device for data logging and post-processing.

25. The textile of claim 20, wherein the system is configured to communicate with at least one of a smart phone and a wireless communication device, wherein at least one of said smart phone and said wireless communication device is an interface for data acquisition and storage, display, and simultaneous relay of data to a cyber infrastructure based cloud computing network for data post processing and storage.

26. The textile of claim 20, wherein said at least one sensor comprises at one of:
a fabric-based or film-based resistive or capacitive electrode, with hierarchically organized nanostructures, for Biopotential sensing;
a plethysmographic optical-reflectance, absorbance and transmittance-array for blood flow monitoring;
a piezoelectric fabric or film based hydrophone sensor enmeshed in the textile for proximal broad spectrum acoustic monitoring for medical diagnostics;
a piezo-resistive fabric or film based strain sensitive sensor enmeshed in the textile for thoracic distention; and
a fabric or film based resistive temperature detector for spatio-temporal body temperature profiling.
